(19) United States
(12) Patent Application Publication
(10) Pub. No.: US 2002/0106738 A1
(43) Pub. Date: Aug. 8, 2002

(54) Novel Siglec gene

(76) Inventors: George Foussias, Toronto (CA); Eleftherios Diamandis, Toronto (CA)

Correspondence Address: MERCHANT & GOULD PC, P.O. BOX 2903, MINNEAPOLIS, MN, 55402-0903, US

(21) Appl. No.: 09/972,714

(22) Filed: Oct. 5, 2001

Related U.S. Application Data
(60) Provisional application No. 60/239,007, filed on Oct. 6, 2000.

(51) Int. Cl. C12Q 1/68; G01N 33/53; A01K 67/00; C07H 21/04; C07K 14/435

(52) U.S. Cl. 435/69.1; 435/320.1; 435/325; 435/6; 435/7.1; 530/350; 536/23.2; 800/8

(57) ABSTRACT

The invention relates to nucleic acid molecules, proteins encoded by such nucleic acid molecules; and use of the proteins and nucleic acid molecules.

### FIELD OF THE INVENTION

The invention relates to nucleic acid molecules, proteins encoded by such nucleic acid molecules; and use of the proteins and nucleic acid molecules

### BACKGROUND OF THE INVENTION

Sialic acid binding immunoglobulin-like lectins (Siglecs) are a family of recently discovered type 1 transmembrane proteins belonging to the immunoglobulin superfamily (IgSF) (1). In addition to their type 1 transmembrane topology, these proteins are characterized by the presence of one N-terminal V-set Ig-like domain, a variable number of downstream C2-set domains, and the ability to bind sialic acid in glycoproteins and glycolipids (2). So far there have been nine Siglec family members described in humans, each with its own unique expression pattern: Siglecl (sialoadhesin) expressed on macrophages (3); Siglec2 (CD22) on B lymphocytes (4); Siglec3 (CD33) on myeloid progenitor cells and monocytes (5); Siglec4a (myelin-associated glycoprotein (MAG)) on oligodendrocytes and Schwann cells (6); Siglec5 on neutrophils (7); Siglec6 on B lymphocytes (8); Siglec7 (p75/AIRM1) on natural killer cells (9, 10); Siglec8 on eosinophils (11); and Siglec9 on neutrophils, monocytes, and various lymphocytes (12-14).

The members of the Siglec family are highly homologous in their extracellular, Ig-like, domains, particularly for the CD33-like subgroup, which includes Siglec3, 5, 6, 7, 8 and 9 (12, 14). This subgroup of genes has been localized to human chromosome 19q13.3-13.4, and it has been suggested that it may be the result of relatively recent gene duplication and exon shuffling (13, 14). In addition to the highly homologous extracellular domains, all the CD33-like Siglecs except Siglec8 show conservation of two cytoplasmic tyrosine-based motifs (11). The first of these contains the consensus sequence for the immunoreceptor tyrosine kinase inhibitory motif (ITIM), (ILV)xYxx(LV) (x being any amino acid) (15, 16). This motif has been shown to be the binding site for the SH2 (src homology 2) domains of the SH2 domain-containing protein tyrosine phosphatases SHP-1 and SHP-2 (17, 18), as well as the SH2 domain-containing inositol phosphatases SHIP1 and SHIP2 (19). The second motif displays homology to a tyrosine-based motif, TxYxx(IV), identified in the signaling lymphocyte activation molecule (SLAM) and was found responsible for its association with the SLAM-associated protein (SAP), which in turn blocks the binding of SHP-2 to phosphorylated SLAM (20, 21).

### SUMMARY OF THE INVENTION

The present inventors have identified the precise genomic region containing the Siglec8 gene. It is located on chromosome 19q13.4, approximately 330 kb downstream of the Siglec9 gene. Further, they have identified a novel Siglec8 variant, named Siglec8-Long (Siglec8-L), which differs in its last two exons from the previously published mRNA sequence of Siglec8 (GenBank accession no. AF195092). Both Siglec8 and Siglec8-L are comprised of seven exons, of which the first five are identical (SEQ ID NO. 2-6), followed by marked differences in exon usage and mRNA splicing. The 499 amino acid protein encoded by the Siglec8-L open reading frame has a molecular weight of 54 kDa. Like the other members of the CD33-like subgroup of Siglecs, except for the previously published Siglec8, Siglec8-L also contains the two tyrosine-based motifs that have been found to recruit both SH2 domain-containing tyrosine and inositol phosphatases.

The siglec8-long protein described herein is referred to as “Siglec8-L Protein”. The gene encoding the protein is referred to as “siglec8-l”.

Broadly stated the present invention relates to an isolated nucleic acid molecule of at least 30 nucleotides which hybridizes to one of SEQ. ID. NOs. 1 to 9, or the complement of one of SEQ ID NOs. 1 to 9, under stringent hybridization conditions.

The invention also contemplates a nucleic acid molecule comprising a sequence encoding a truncation of a SIGLEC8-L Protein, an analog, or a homolog of a SIGLEC8-L Protein or a 2 O truncation thereof. (SIGLEC8-L Protein and truncations, analogs and homologs of SIGLEC8-L Protein are also collectively referred to herein as “SIGLEC8-L Related Proteins”).

The nucleic acid molecules of the invention may be inserted into an appropriate expression vector, i.e. a vector that contains the necessary elements for the transcription and translation of the inserted coding sequence. Accordingly, recombinant expression vectors adapted for transformation of a host cell may be constructed which comprise a nucleic acid molecule of the invention and one or more transcription and translation elements linked to the nucleic acid molecule.

The recombinant expression vector can be used to prepare transformed host cells expressing SIGLEC8-L Related Proteins. Therefore, the invention further provides host cells containing a recombinant molecule of the invention. The invention also contemplates transgenic non-human mammals whose germ cells and somatic cells contain a recombinant molecule comprising a nucleic acid molecule of the invention, in particular one which encodes an analog of the SIGLEC8-L Protein, or a truncation of the SIGLEC8-L Protein.

The invention further provides a method for preparing SIGLEC8-L Related Proteins utilizing the purified and isolated nucleic acid molecules of the invention. In an embodiment a method for preparing a SIGLEC8-L Related Protein is provided comprising (a) transferring a recombinant expression vector of the invention into a host cell; (b) selecting transformed host cells from untransformed host cells; (c) culturing a selected transformed host cell under conditions which allow expression of the SIGLEC8-L Related Protein; and (d) isolating the SIGLEC8-L Related Protein.

The invention further broadly contemplates an isolated SIGLEC8-L Protein comprising an amino acid sequence of SEQ.ID.NO. 10.

The SIGLEC8-L Related Proteins of the invention may be conjugated with other molecules, such as proteins, to prepare fusion proteins. This may be accomplished, for example, by the synthesis of N-terminal or C-terminal fusion proteins.

The invention further contemplates antibodies having specificity against an epitope of a SIGLEC8-L Related Protein of the invention. Antibodies may be labeled with a detectable substance and used to detect proteins of the invention in tissues and cells. Antibodies may have particular use in therapeutic applications, for example to react with tumor cells, and in conjugates and immunotoxins as target selective carriers of various agents which have antitumor effects including chemotherapeutic drugs, growth factors, cytokines, toxins, immunological response modifiers, enzymes, and radioisotopes.

The invention also permits the construction of nucleotide probes which are unique to the nucleic acid molecules of the invention and/or to proteins of the invention. Therefore, the invention also relates to a probe comprising a nucleic acid sequence of the invention, or a nucleic acid sequence encoding a protein of the invention, or a part thereof. The probe may be labeled, for example, with a detectable substance and it may be used to select from a mixture of nucleotide sequences a nucleic acid molecule of the invention including nucleic acid molecules coding for a protein which displays one or more of the properties of a protein of the invention. A probe may be used to mark tumors.

The invention also provides antisense nucleic acid molecules e.g. by production of a mRNA or DNA strand in the reverse orientation to a sense molecule. An antisense nucleic acid molecule may be used to suppress the growth of a SIGLEC8-L expressing (e.g. cancerous) cell.

The invention still further provides a method for identifying a substance which binds to a protein of the invention comprising reacting the protein with at least one substance which potentially can bind with the protein, under conditions which permit the formation of complexes between the substance and protein and detecting binding. Binding may be detected by assaying for complexes, for free substance, or for non-complexed protein. The invention also contemplates methods for identifying substances that bind to other intracellular proteins that interact with a SIGLEC8-L Related Protein. Methods can also be utilized which identify compounds which bind to SIGLEC8-L gene regulatory sequences (e.g. promoter sequences).

Still further the invention provides a method for evaluating a compound for its ability to modulate the biological activity of a SIGLEC8-L Related Protein of the invention. For example, a substance which inhibits or enhances the interaction of the protein and a substance which binds to the protein may be evaluated. In an embodiment, the method comprises providing a known concentration of a SIGLEC8-L Related Protein, with a substance which binds to the protein and a test compound under conditions which permit the formation of complexes between the substance and protein, and removing and/or detecting complexes.

Compounds which modulate the biological activity of a protein of the invention may also be identified using the methods of the invention by comparing the pattern and level of expression of the protein of the invention in tissues and cells, in the presence, and in the absence of the compounds.

The proteins of the invention, antibodies, antisense nucleic acid molecules, and substances and compounds identified using the methods of the invention, and peptides of the invention may be used to modulate the biological activity of a SIGLEC8-L Related Protein of the invention, and they may be used in the treatment of conditions associated with a SIGLEC8-L Related Protein such as cancer and hematopoietic disorders. Accordingly, the substances and compounds may be formulated into compositions for administration to individuals suffering from such conditions. In particular, the antibodies, antisense nucleic acid molecules, substances and compounds may be used to treat patients who have a SIGLEC8-L Related Protein in, or on, their cancer cells.

Therefore, the present invention also relates to a composition comprising one or more of a protein of the invention, or a substance or compound identified using the methods of the invention, and a pharmaceutically acceptable carrier, excipient or diluent. A method for treating or preventing a condition associated with a SIGLEC8-L Related Protein (e.g. hematopoietic disorders or cancer) is also provided comprising administering to a patient in need thereof, a SIGLEC8-L Related Protein of the invention, or a composition of the invention.

Another aspect of the invention is the use of a SIGLEC8-L Related Protein, peptides derived therefrom, or chemically produced (synthetic) peptides, or any combination of these molecules, for use in the preparation of vaccines to prevent cancer and/or to treat cancer, in particular to prevent and/or treat cancer in patients who have a SLG Related Protein detected on their cells. These vaccine preparations may also be used to prevent patients from having tumors prior to their occurrence.

The invention broadly contemplates vaccines for stimulating or enhancing in a subject to whom the vaccine is administered production of antibodies directed against a SIGLEC8-L Related Protein.

The invention also provides a method for stimulating or enhancing in a subject production of antibodies directed against a SIGLEC8-L Related Protein. The method comprises administering to the subject a vaccine of the invention in a dose effective for stimulating or enhancing production of the antibodies.

The invention further provides methods for treating, preventing, or delaying recurrence of cancer. The methods comprise administering to the subject a vaccine of the invention in a dose effective for treating, preventing, or delaying recurrence of cancer.

In other embodiments, the invention provides a method for identifying inhibitors of a SIGLEC8-L Related Protein interaction, comprising

(a) providing a reaction mixture including the SLG Related Protein and a substance that binds to the SIGLEC8-L Related Protein, or at least a portion of each which interact;

(b) contacting the reaction mixture with one or more test compounds;

(c) identifying compounds which inhibit the interaction of the SIGLEC8-L Related Protein and substance.

In certain preferred embodiments, the reaction mixture is a whole cell. In other embodiments, the reaction mixture is a cell lysate or purified protein composition. The subject method can be carried out using libraries of test compounds. Such agents can be proteins, peptides, nucleic acids, carbohydrates, small organic molecules, and natural product extract libraries, such as those isolated from animals, plants, fungus and/or microbes.

Still another aspect of the present invention provides a method of conducting a drug discovery business comprising:

(a) providing one or more assay systems for identifying agents by their ability to inhibit or potentiate the interaction of a SIGLEC8-L Related Protein and a substance that binds to the protein;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1: Genomic Organization of the Siglec8 Gene. Based on experimental results, as well as the previously published sequence for the Siglec8 mRNA (GenBank Accession No. AF195092), it was established that both Siglec8 and the Siglec8-L are composed of seven exons (Arabic numerals). The two mRNA species are identical until exon six (SEQ ID NO. 7), where the Siglec8 mRNA contains the whole of exon six (6a & 6b) and continues to exon 7a, (SEQ ID NO 8) indicated by the broken line, while the Siglec8-L mRNA contains only exons 6b and 7b, shown by the solid line. The location of the stop codon (TGA) is shown for both mRNA species, and differs due to a change in the open reading frame. Splice sites are conserved (-mGT . . . AGm-, where m is any base) in Siglec8-L between exons 6b and 7b, but not between exons 6a and 7a in the Siglec8 sequence reported by Floyd et. al. (11).

FIG. 2: Protein Sequence Alignment for the Siglec3-like Subgroup of Siglecs. The sequence of Siglec8-L was aligned to those of Siglec8, as well as the remaining members of the Siglec3-like subgroup of Siglecs, using the ClustalX multiple alignment tool (25). The solid vertical lines indicate the positions of the exon boundaries. In all but one case, shown by the broken vertical line, the exon boundaries match those found for Siglec9 (12). The conserved cysteine residues responsible for the intra- and interdomain disufide bonds (2, 36, 37) are indicated by the star (* ), while the triangles (▾) denote the aromatic residues believed to be important for sialic acid binding, based on findings for Siglec1 (sialoadhesin) (38). The signal peptide cleavage site for Siglec8-L, indicated by the solid circle (●), was predicted using the SignalP program (39). The Ig-like domain assignments, as well as those for the transmembrane and cytoplasmic domains, are based on previous reports (11) and the one domain-one exon rule (40). The positions of the two tyrosine-based motifs, ITIM and SLAM-like, are indicated. The GenBank accession numbers are as follows: Siglec8-L: AF287892; Siglec8: AF195092; Siglec7: AF170485; Siglec6: NM 001245; Siglec5: NM_{—}003830; Siglec3 (CD33): M23197.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y); DNA Cloning: A Practical Approach, Volumes I and II (D.N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

1. Nucleic Acid Molecules of the Invention

As hereinbefore mentioned, the invention provides an isolated nucleic acid molecule having a sequence encoding a SIGLEC8-L Related Protein. The term “isolated” refers to a nucleic acid substantially free of cellular material or culture medium when produced by recombinant DNA techniques, or chemical reactants, or other chemicals when chemically synthesized. An “isolated” nucleic acid may also be free of sequences which naturally flank the nucleic acid (i.e., sequences located at the 5′ and 3′ ends of the nucleic acid molecule) from which the nucleic acid is derived. The term “nucleic acid” is intended to include DNA and RNA and can be either double stranded or single stranded. In an embodiment, a nucleic acid molecule encodes a SIGLEC8-L Related Protein comprising an amino acid sequence of SEQ.ID.NO. 10, preferably a nucleic acid molecule comprising a nucleic acid sequence of one of SEQ.ID.NOs. 1, 7, 8, or 9.

In an embodiment, the invention provides an isolated nucleic acid molecule which comprises:

(i) a nucleic acid sequence encoding a protein having substantial sequence identity with an amino acid sequence of SEQ.ID.NO. 10;

(ii) a nucleic acid sequence encoding a protein comprising an amino acid sequence of SEQ.ID.NO. 10;

(iii) nucleic acid sequences complementary to (i);

(iv) a degenerate form of a nucleic acid sequence of (i);

(v) a nucleic acid sequence capable of hybridizing under stringent conditions to a nucleic acid sequence in (i), (ii) or (iii);

(vi) a nucleic acid sequence encoding a truncation, an analog, an allelic or species variation of a protein comprising an amino acid sequence of SEQ.ID.NO. 10; or

(vii) a fragment, or allelic or species variation of (i), (ii) or (iii).

Preferably, a purified and isolated nucleic acid molecule of the invention comprises:

(i) a nucleic acid sequence comprising the sequence of one of SEQ.ID.NOs. 1, 7, 8, or 9 wherein T can also be U;

(ii) nucleic acid sequences complementary to (i), preferably complementary to the full nucleic acid sequence of one of SEQ.ID.NOs.1, 7, 8, or 9;

(iii) a nucleic acid capable of hybridizing under stringent conditions to a nucleic acid of (i) or (ii) and preferably having at least 18 nucleotides; or

(iv) a nucleic acid molecule differing from any of the nucleic acids of (i) to (iii) in codon sequences due to the degeneracy of the genetic code.

The invention includes nucleic acid sequences complementary to a nucleic acid encoding a protein comprising an amino acid sequence of SEQ.ID.NO. 10, preferably the nucleic acid sequences complementary to a full nucleic acid sequence of one of SEQ.ID.NOs. 1, 7, 8, or 9.

The invention includes nucleic acid molecules having substantial sequence identity or homology to nucleic acid sequences of the invention or encoding proteins having substantial identity or similarity to the amino acid sequence of SEQ.ID.NO. 10. Preferably, the nucleic acids have substantial sequence identity for example at least 65%, 70%, 75%, 80%, or 85% nucleic acid identity; more preferably 90% nucleic acid identity; and most preferably at least 95%, 96%, 97%, 98%, or 99% sequence identity. “Identity” as known in the art and used herein, is a relationship between two or more amino acid sequences or two or more nucleic acid sequences, as determined by comparing the sequences. It also refers to the degree of sequence relatedness between amino acid or nucleic acid sequences, as the case may be, as determined by the match between strings of such sequences. Identity and similarity are well known terms to skilled artisans and they can be calculated by conventional methods (for example see Computational Molecular Biology, Lesk, A. M. ed., Oxford University Press, New York, 1988; Biocomputing: Informatics and Genome Projects, Smith, D. W. ed., Academic Press, New York, 1993; Computer Analysis of Sequence Data, Part I, Griffin, A. M. and Griffin, H. G. eds., Humana Press, New Jersey, 1994; Sequence Analysis in Molecular Biology, von Heinje, G. Acadmeic Press, 1987; and Sequence Analysis Primer, Gribskov, M. and Devereux, J. eds. M. Stockton Press, New York, 1991, Carillo, H. and Lipman, D., SIAM J. Applied Math. 48:1073, 1988). Methods which are designed to give the largest match between the sequences are generally preferred. Methods to determine identity and similarity are codified in publicly available computer programs including the GCG program package (Devereux J. et al., Nucleic Acids Research 12(1): 387, 1984); BLASTP, BLASTN, and FASTA (Atschul, S.F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990).

Isolated nucleic acid molecules encoding a SIGLEC8-L Protein, and having a sequence which differs from a nucleic acid sequence of the invention due to degeneracy in the genetic code are also within the scope of the invention. Such nucleic acids encode functionally equivalent proteins (e.g. a SIGLEC8-L Protein) but differ in sequence from the sequence of a SIGLEC8-L Protein due to degeneracy in the genetic code. As one example, DNA sequence polymorphisms within the nucleotide sequence of a SIGLEC8-L Protein may result in silent mutations which do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of a SIGLEC8-L Protein. These amino acid polymorphisms are also within the scope of the present invention.

Another aspect of the invention provides a nucleic acid molecule which hybridizes under stringent conditions, preferably high stringency conditions to a nucleic acid molecule which comprises a sequence which encodes a SIGLEC8-L Protein having an amino acid sequence shown in SEQ.ID.NO. 10. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0 x sodium chloride/sodium citrate (SSC) at about 45° C., followed by a wash of 2.0× SSC at 50° C. may be employed. The stringency may be selected based on the conditions used in the wash step. By way of example, the salt concentration in the wash step can be selected from a high stringency of about 0.2× SSC at 50° C. In addition, the temperature in the wash step can be at high stringency conditions, at about 65° C.

It will be appreciated that the invention includes nucleic acid molecules encoding a SIGLEC8-L Related Protein including truncations of a SIGLEC8-L Protein, and analogs of a SIGLEC8-L Protein as described herein. It will further be appreciated that variant forms of the nucleic acid molecules of the invention which arise by alternative splicing of an mRNA corresponding to a cDNA of the invention are encompassed by the invention.

An isolated nucleic acid molecule of the invention which comprises DNA can be isolated by preparing a labelled nucleic acid probe based on all or part of a nucleic acid sequence of the invention. The labeled nucleic acid probe is used to screen an appropriate DNA library (e.g. a cDNA or genomic DNA library). For example, a cDNA library can be used to isolate a cDNA encoding a SIGLEC8-L Related Protein by screening the library with the labeled probe using standard techniques. Alternatively, a genomic DNA library can be similarly screened to isolate a genomic clone encompassing a gene encoding a SIGLEC8-L Related Protein. Nucleic acids isolated by screening of a cDNA or genomic DNA library can be sequenced by standard techniques.

An isolated nucleic acid molecule of the invention which is DNA can also be isolated by selectively amplifying a nucleic acid encoding a SIGLEC8-L Related Protein using the polymerase chain reaction (PCR) methods and cDNA or genomic DNA. It is possible to design synthetic oligonucleotide primers from the nucleotide sequence of the invention for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a SIGLEC8-L Related Protein into an appropriate vector which allows for transcription of the cDNA to produce an RNA molecule which encodes a SIGLEC8-L Related Protein. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

Nucleic acid molecules of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule encodes a SIGLEC8-L Related Protein can be accomplished by expressing the cDNA in an appropriate host cell by standard techniques, and testing the expressed protein in the methods described herein. A cDNA encoding a SIGLEC8-L Related Protein can be sequenced by standard techniques, such as dideoxynucleotide chain termination or Maxam-Gilbert chemical sequencing, to determine the nucleic acid sequence and the predicted amino acid sequence of the encoded protein.

The initiation codon and untranslated sequences of a SIGLEC8-L Related Protein may be determined using computer software designed for the purpose, such as PC/Gene (IntelliGenetics Inc., Calif.). The intron-exon structure and the transcription regulatory sequences of a gene encoding a SIGLEC8-L Related Protein may be confirmed by using a nucleic acid molecule of the invention encoding a SIGLEC8-L Related Protein to probe a genomic DNA clone library. Regulatory elements can be identified using standard techniques. The function of the elements can be confirmed by using these elements to express a reporter gene such as the lacZ gene which is operatively linked to the elements. These constructs may be introduced into cultured cells using conventional procedures or into non-human transgenic animal models. In addition to identifying regulatory elements in DNA, such constructs may also be used to identify nuclear proteins interacting with the elements, using techniques known in the art.

In a particular embodiment of the invention, the nucleic acid molecules isolated using the methods described herein are mutant Siglec8-L gene alleles. The mutant alleles may be isolated from individuals either known or proposed to have a genotype which contributes to the symptoms of a disorder (e.g. cancer). Mutant alleles and mutant allele products may be used in therapeutic and diagnostic methods described herein. For example, a cDNA of a mutant Siglec8-L gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an individual suspected of or known to carry a mutant allele, or a cDNA library can be constructed using RNA from tissue known, or suspected to express the mutant allele. A nucleic acid encoding a normal Siglec8-L gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant Siglec8-L allele. Gene products made by the putatively mutant tissue may be expressed and screened, for example using antibodies specific for a SIGLEC8-L Related Protein as described herein. Library clones identified using the antibodies can be purified and subjected to sequence analysis.

The sequence of a nucleic acid molecule of the invention, or a fragment of the molecule, may be inverted relative to its normal presentation for transcription to produce an antisense nucleic acid molecule. An antisense nucleic acid molecule may be constructed using chemical synthesis and enzymatic ligation reactions using procedures known in the art.

2. Proteins of the Invention

An amino acid sequence of a SIGLEC8-L Protein comprises a sequence as shown in SEQ.ID.NO. 10.

In addition to proteins comprising an amino acid sequence as shown in SEQ.ID.NO. 10, the proteins of the present invention include truncations of a SIGLEC8-L Protein, analogs of a SIGLEC8-L Protein, and proteins having sequence identity or similarity to a SIGLEC8-L Protein, and truncations thereof as described herein (i.e. SIGLEC8-L Related Proteins). Truncated proteins may comprise peptides of between 3 and 70 amino acid residues, ranging in size from a tripeptide to a 70 mer polypeptide.

The truncated proteins may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated proteins may have a carboxyl group, an amido group, a T-butyloxycarbonyl group, or a macromolecule including but not limited to lipid-fatty acid conjugates, polyethylene glycol, or carbohydrates at the carboxy terminal end.

The proteins of the invention may also include analogs of a SIGLEC8-L Protein, and/or truncations thereof as described herein, which may include, but are not limited to a SIGLEC8-L Protein, containing one or more amino acid substitutions, insertions, and/or deletions. Amino acid substitutions may be of a conserved or non-conserved nature. Conserved amino acid substitutions involve replacing one or more amino acids of a SIGLEC8-L Protein amino acid sequence with amino acids of similar charge, size, and/or hydrophobicity characteristics. When only conserved substitutions are made the resulting analog is preferably functionally equivalent to a SIGLEC8-L Protein. Non-conserved substitutions involve replacing one or more amino acids of the SIGLEC8-L Protein amino acid sequence with one or more amino acids which possess dissimilar charge, size, and/or hydrophobicity characteristics.

One or more amino acid insertions may be introduced into a SIGLEC8-L Protein. Amino acid insertions may consist of single amino acid residues or sequential amino acids ranging from 2 to 15 amino acids in length.

Deletions may consist of the removal of one or more amino acids, or discrete portions from a SIGLEC8-L Protein sequence. The deleted amino acids may or may not be contiguous. The lower limit length of the resulting analog with a deletion mutation is about 10 amino acids, preferably 20 to 40 amino acids.

The proteins of the invention include proteins with sequence identity or similarity to a SIGLEC8-L Protein and/or truncations thereof as described herein. Such SIGLEC8-L Proteins include proteins whose amino acid sequences are comprised of the amino acid sequences of SIGLEC8-L Protein regions from other species that hybridize under selected hybridization conditions (see discussion of stringent hybridization conditions herein) with a probe used to obtain a SIGLEC8-L Protein. These proteins will generally have the same regions which are characteristic of a SIGLEC8-L Protein. Preferably a protein will have substantial sequence identity for example, about 65%, 70%, 75%, 80%, or 85% identity, preferably 90% identity, more preferably at least 95%, 96%, 97%, 98%, or 99% identity, and most preferably 98% identity with an amino acid sequence shown in in SEQ.ID.NO. 10. A percent amino acid sequence homology, similarity or identity is calculated as the percentage of aligned amino acids that match the reference sequence using known methods as described herein.

The invention also contemplates isoforms of the proteins of the invention. An isoform contains the same number and kinds of amino acids as a protein of the invention, but the isoform has a different molecular structure. Isoforms contemplated by the present invention preferably have the same properties as a protein of the invention as described herein.

The present invention also includes SIGLEC8-L Related Proteins conjugated with a selected protein, or a marker protein (see below) to produce fusion proteins. Additionally, immunogenic portions of a SIGLEC8-L Protein and a SIGLEC8-L Protein Related Protein are within the scope of the invention.

A SIGLEC8-L Related Protein of the invention may be prepared using recombinant DNA methods. Accordingly, the nucleic acid molecules of the present invention having a sequence which encodes a SIGLEC8-L Related Protein of the invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used.

The invention therefore contemplates a recombinant expression vector of the invention containing a nucleic acid molecule of the invention, and the necessary regulatory sequences for the transcription and translation of the inserted protein-sequence. Suitable regulatory sequences may be derived from a variety of sources, including bacterial, fungal, viral, mammalian, or insect genes [For example, see the regulatory sequences described in Goeddel, Gene Expression Technology: Methods in Enzymology 185, Academic Press, San Diego, Calif. (1990)]. Selection of appropriate regulatory sequences is dependent on the host cell chosen as discussed below, and may be readily accomplished by one of ordinary skill in the art. The necessary regulatory sequences may be supplied by the native SIGLEC8-L Protein and/or its flanking regions.

The invention further provides a recombinant expression vector comprising a DNA nucleic acid molecule of the invention cloned into the expression vector in an antisense orientation. That is, the DNA molecule is linked to a regulatory sequence in a manner which allows for expression, by transcription of the DNA molecule, of an RNA molecule which is antisense to the nucleic acid sequence of a protein of the invention or a fragment thereof. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The recombinant expression vectors of the invention may also contain a marker gene which facilitates the selection of host cells transformed or transfected with a recombinant molecule of the invention. Examples of marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

The recombinant expression vectors may be introduced into host cells to produce a transformant host cell. “Transformant host cells” include host cells which have been transformed or transfected with a recombinant expression vector of the invention. The terms “transformed with”, “transfected with”, “transformation” and “transfection” encompass the introduction of a nucleic acid (e.g. a vector) into a cell by one of many standard techniques. Prokaryotic cells can be transformed with a nucleic acid by, for example, electroporation or calcium-chloride mediated transformation. A nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g. cleaves) the protein in a desired fashion. Host systems or cell lines may be selected which have specific and characteristic mechanisms for post-translational processing and modification of proteins. For example, eukaryotic host cells including CHO, VERO, BHK, HeLA, COS, MDCK, 293, 3T3, and W138 may be used. For long-term high-yield stable expression of the protein, cell lines and host systems which stably express the gene product may be engineered.

Host cells and in particular cell lines produced using the methods described herein may be particularly useful in screening and evaluating compounds that modulate the activity of a SIGLEC8-L Related Protein.

The proteins of the invention may also be expressed in non-human transgenic animals including but not limited to mice, rats, rabbits, guinea pigs, micro-pigs, goats, sheep, pigs, non-human primates (e.g. baboons, monkeys, and chimpanzees) [see Hammer et al. (Nature 315:680-683, 1985), Palmiter et al. (Science 222:809-814, 1983), Brinster et al. (Proc Natl. Acad. Sci USA 82:44384442, 1985), Palmiter and Brinster (Cell. 41:343-345, 1985) and U.S. Pat. No. 4,736,866)]. Procedures known in the art may be used to introduce a nucleic acid molecule of the invention encoding a SIGLEC8-L Related Protein into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries the SIGLEC8-L gene in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant SIGLEC8-L Related Protein in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissue may also be evaluated immunocytochemically using antibodies against SIGLEC8-L Protein.

Proteins of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

N-terminal or C-terminal fusion proteins comprising a SIGLEC8-L Related Protein of the invention conjugated with other molecules, such as proteins, may be prepared by fusing, through recombinant techniques, the N-terminal or C-terminal of a SIGLEC8-L Related Protein, and the sequence of a selected protein or marker protein with a desired biological function. The resultant fusion proteins contain SIGLEC8-L Protein fused to the selected protein or marker protein as described herein. Examples of proteins which may be used to prepare fusion proteins include immunoglobulins, glutathione-S-transferase (GST), hemagglutinin (HA), and truncated myc.

3. Antibodies

SIGLEC8-L Related Proteins of the invention can be used to prepare antibodies specific for the proteins. Antibodies can be prepared which bind a distinct epitope in an unconserved region of the protein. An unconserved region of the protein is one that does not have substantial sequence homology to other proteins. A region from a conserved region such as a well-characterized domain can also be used to prepare an antibody to a conserved region of a SIGLEC8-L Related Protein. Antibodies having specificity for a SIGLEC8-L Related Protein may also be raised from fusion proteins created by expressing fusion proteins in bacteria as described herein.

The invention can employ intact monoclonal or polyclonal antibodies, and immunologically active fragments (e.g. a Fab, (Fab)2 fragment, or Fab expression library fragments and epitope-binding fragments thereof), humanized antibodies, an antibody heavy chain, and antibody light chain, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

4. Applications of the Nucleic Acid Molecules, SIGLEC8-L Related Proteins, and Antibodies of the Invention

The nucleic acid molecules, SIGLEC8-L Related Proteins, and antibodies of the invention may be used in the prognostic and diagnostic evaluation of conditions associated with a SIGLEC8-L Related Protein such as cancer and hematopoietic disorders, and the identification of subjects with a predisposition to such conditions (Section 4.1.1 and 4.1.2).

In an embodiment of the invention, a method is provided for detecting the expression of the marker SIGLEC8-L in a patient comprising:

(a) taking a sample derived from a patient; and

(b) detecting in the sample a nucleic acid sequence encoding SIGLEC8-L or a protein product encoded by a SIGLEC8-L nucleic acid sequence.

In a particular embodiment of the invention, the nucleic acid molecules, SIGLEC8-L Related Proteins, and antibodies of the invention may be used in the diagnosis and staging of cancer.

Methods for detecting nucleic acid molecules and SIGLEC8-L Related Proteins of the invention, can be used to monitor conditions such as cancer by detecting SIGLEC8-L Related Proteins and nucleic acid molecules encoding SIGLEC8-L Related Proteins. The applications of the present invention also include methods for the identification of compounds that modulate the biological activity of SIGLEC8-L or SIGLEC8-L Related Proteins (Section 4.2). The compounds, antibodies etc. may be used for the treatment of conditions associated with a SIGLEC8-L Related Protein such as cancer (Section 4.3). It would also be apparent to one skilled in the art that the methods described herein may be used to study the developmental expression of SIGLEC8-L Related Proteins and, accordingly, will provide further insight into the role of SIGLEC8-L Related Proteins.

4.1 Diagnostic Methods

A variety of methods can be employed for the diagnostic and prognostic evaluation of conditions associated with a SIGLEC8-L Related Protein such as cancer, and the identification of subjects with a predisposition to such conditions. Such methods may, for example, utilize nucleic acid molecules of the invention, and fragments thereof, and antibodies directed against SIGLEC8-L Related Proteins, including peptide fragments. In particular, the nucleic acids and antibodies may be used, for example, for: (1) the detection of the presence of SIGLEC8-L mutations, or the detection of either over- or under-expression of SIGLEC8-L mRNA relative to a non-disorder state or the qualitative or quantitative detection of alternatively spliced forms of SIGLEC8-L transcripts which may correlate with certain conditions or susceptibility toward such conditions; and (2) the detection of either an over- or an under-abundance of SIGLEC8-L Related Proteins relative to a non- disorder state or the presence of a modified (e.g., less than full length) SIGLEC8-L Protein which correlates with a disorder state, or a progression toward a disorder state.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein may be performed by utilizing pre-packaged diagnostic kits comprising at least one specific SIGLEC8-L nucleic acid or antibody described herein, which may be conveniently used, e.g., in clinical settings, to screen and diagnose patients and to screen and identify those individuals exhibiting a predisposition to developing a disorder.

Nucleic acid-based detection techniques are described, below, in Section 4.1.1. Peptide detection techniques are described, below, in Section 4.1.2. The samples that may be analyzed using the methods of the invention include those which are known or suspected to express SIGLEC8-L or contain SIGLEC8-L Related Proteins. The samples may be derived from a patient or a cell culture, and include but are not limited to biological fluids, tissue extracts, freshly harvested cells, and lysates of cells which have been incubated in cell cultures.

Oligonucleotides or longer fragments derived from any of the nucleic acid molecules of the invention may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

4.1.1 Methods for Detecting Nucleic Acid Molecules of the Invention

The nucleic acid molecules of the invention allow those skilled in the art to construct nucleotide probes for use in the detection of nucleic acid sequences of the invention in samples.

Suitable probes include nucleic acid molecules based on nucleic acid sequences encoding at least 5 sequential amino acids from regions of the SIGLEC8-L Protein, preferably they comprise 15 to 30 nucleotides. A nucleotide probe may be labeled with a detectable substance such as a radioactive label which provides for an adequate signal and has sufficient half-life such as 32P, 3H, 14C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect genes, preferably in human cells, that encode SIGLEC8-L Related Proteins. The nucleotide probes may also be useful in the diagnosis of a condition associated with SIGLEC8-L such as cancer; in monitoring the progression of the condition; or monitoring a therapeutic treatment.

The probe may be used in hybridization techniques to detect genes that encode SIGLEC8-L Related Proteins. The technique generally involves contacting and incubating nucleic acids (e.g. recombinant DNA molecules, cloned genes) obtained from a sample from a patient or other cellular source with a probe of the present invention under conditions favorable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules of the invention may involve the amplification of specific gene sequences using an amplification method such as PCR, followed by the analysis of the amplified molecules using techniques known to those skilled in the art. Suitable primers can be routinely designed by one of skill in the art.

Genomic DNA may be used in hybridization or amplification assays of biological samples to detect abnormalities involving SIGLEC8-L structure, including point mutations, insertions, deletions, and chromosomal rearrangements. For example, direct sequencing, single stranded conformational polymorphism analyses, heteroduplex analysis, denaturing gradient gel electrophoresis, chemical mismatch cleavage, and oligonucleotide hybridization may be utilized.

Genotyping techniques known to one skilled in the art can be used to type polymorphisms that are in close proximity to the mutations in a Siglec8-L gene. The polymorphisms may be used to identify individuals in families that are likely to carry mutations. If a polymorphism exhibits linkage disequalibrium with mutations in a Siglec8-L gene, it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

Hybridization and amplification techniques described herein may be used to assay qualitative and quantitative aspects of Siglec8-L expression. For example, RNA may be isolated from a cell type or tissue known to express Siglec8-L and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively splice transcripts detected in normal individuals relative to those individuals exhibiting symptoms of a hematopoietic disorder or other disease conditions.

The primers and probes may be used in the above described methods in situ i.e directly on tissue sections (fixed and/or frozen) of patient tissue obtained from biopsies or resections.

4.1.2 Methods for Detecting SIGLEC8-L Related Proteins

Antibodies specifically reactive with a SIGLEC8-L Related Protein, or derivatives, such as enzyme conjugates or labeled derivatives, may be used to detect SIGLEC8-L Related Proteins in various samples (e.g. biological materials). They may be used as diagnostic or prognostic reagents and they may be used to detect abnormalities in the level of SIGLEC8-L Related Protein expression, or abnormalities in the structure, and/or temporal, tissue, cellular, or subcellular location of a SIGLEC8-L Related Protein. Antibodies may also be used to screen potentially therapeutic compounds in vitro to determine their effects on conditions including cancer. In vitro immunoassays may also be used to assess or monitor the efficacy of particular therapies. The antibodies of the invention may also be used in vitro to determine the level of Siglec8-L expression in cells genetically engineered to produce a SIGLEC8-L Related Protein.

The antibodies may be used in any known immunoassays which rely on the binding interaction between an antigenic determinant of a SIGLEC8-L Related Protein and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify SIGLEC8-L Related Proteins in a sample in order to determine its role in particular cellular events or pathological states, and to diagnose and treat such pathological states.

In particular, the antibodies of the invention may be used in immuno-histochemical analyses, for example, at the cellular and sub-subcellular level, to detect a SIGLEC8-L Related Protein, to localize it to particular cells and tissues, and to specific subcellular locations, and to quantitate the level of expression.

Cytochemical techniques known in the art for localizing antigens using light and electron microscopy may be used to detect a SIGLEC8-L Related Protein. Generally, an antibody of the invention may be labeled with a detectable substance and a SIGLEC8-L Related Protein may be localised in tissues and cells based upon the presence of the detectable substance. Examples of detectable substances include, but are not limited to, the following: radioisotopes (e.g., 3 H, 14C, 35S, 125I, 131I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a SIGLEC8-L Related Protein. By way of example, if the antibody having specificity against a SIGLEC8-L Related Protein is a rabbit IgG antibody, the second antibody may be goat anti-rabbit gamma-globulin labeled with a detectable substance as described herein.

Where a radioactive label is used as a detectable substance, a SIGLEC8-L Related Protein may be localized by radioautography. The results of radioautography may be quantitated by determining the density of particles in the radioautographs by various optical methods, or by counting the grains.

In an embodiment, the invention contemplates a method for monitoring the progression of a condition associated with a SIGLEC8-L Related Protein (e.g. cancer or a hematopoietic disorder) in an individual, comprising:

(a) contacting an amount of an antibody which binds to a SIGLEC8-L Related Protein, with a sample from the individual so as to form a binary complex comprising the antibody and SIGLEC8-L Related Protein in the sample;

(b) determining or detecting the presence or amount of complex formation in the sample;

(c) repeating steps (a) and (b) at a point later in time; and

(d) comparing the result of step (b) with the result of step (c), wherein a difference in the amount of complex formation is indicative of the progression of the condition in said individual.

The amount of complexes may also be compared to a value representative of the amount of the complexes from an individual not at risk of, or afflicted with, the condition.

4.2 Methods for Identifying or Evaluating Substances/Compounds

The methods described herein are designed to identify substances that modulate the biological activity of a SIGLEC8-L Related Protein including substances that bind to SIGLEC8-L Related Proteins, or bind to other proteins that interact with a SIGLEC8-L Related Protein, to compounds that interfere with, or enhance the interaction of a SIGLEC8-L Related Protein and substances that bind to the SIGLEC8-L Related Protein or other proteins that interact with a SIGLEC8-L Related Protein. Methods are also utilized that identify compounds that bind to SIGLEC8-L regulatory sequences.

The substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)2, and Fab expression library fragments, and epitope-binding fragments thereof)], and small organic or inorganic molecules. The substance or compound may be an endogenous physiological compound or it may be a natural or synthetic compound.

Substances which modulate a SIGLEC8-L Related Protein can be identified based on their ability to bind to a SIGLEC8-L Related Protein. Therefore, the invention also provides methods for identifying substances which bind to a SIGLEC8-L Related Protein. Substances identified using the methods of the invention may be isolated, cloned and sequenced using conventional techniques. A substance that associates with a polypeptide of the invention may be an agonist or antagonist of the biological or immunological activity of a polypeptide of the invention.

The term “agonist” refers to a molecule that increases the amount of, or prolongs the duration of, the activity of the protein. The term “antagonist” refers to a molecule which decreases the biological or immunological activity of the protein. Agonists and antagonists may include proteins, nucleic acids, carbohydrates, or any other molecules that associate with a protein of the invention.

Substances which can bind with a SIGLEC8-L Related Protein may be identified by reacting 1 a SIGLEC8-L Related Protein with a test substance which potentially binds to a SIGLEC8-L Related Protein, under conditions which permit the formation of substance-SIGLEC8-L Related Protein complexes, and removing and/or detecting the complexes. The complexes can be detected by assaying for substance-SIGLEC8-L Related Protein complexes, for free substance, or for non-complexed SIGLEC8-L Related Protein. Conditions which permit the formation of substance-SIGLEC8-L Related Protein complexes may be selected having regard to factors such as the nature and amounts of the substance and the protein.

The substance-protein complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against SIGLEC8-L Related Protein or the substance, or labeled SIGLEC8-L Related Protein, or a labeled substance may be utilized. The antibodies, proteins, or substances may be labeled with a detectable substance as described above.

A SIGLEC8-L Related Protein, or the substance used in the method of the invention may be insolubilized. For example, a SIGLEC8-L Related Protein, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling.

The invention also contemplates a method for evaluating a compound for its ability to modulate the biological activity of a SIGLEC8-L Related Protein of the invention, by assaying for an agonist or antagonist (i.e. enhancer or inhibitor) of the binding of a SIGLEC8-L Related Protein with a substance which binds with a SIGLEC8-L Related Protein. Examples of such substances include SH2 domain-containing tyrosine and inositol phosphatases. The basic method for evaluating if a compound is an agonist or antagonist of the binding of a SIGLEC8-L Related Protein and a substance that binds to the protein, is to prepare a reaction mixture containing the SIGLEC8-L Related Protein and the substance under conditions which permit the formation of substance-SIGLEC8-L Related Protein complexes, in the presence of a test compound. The test compound may be initially added to the mixture, or may be added subsequent to the addition of the SIGLEC8-L Related Protein and substance. Control reaction mixtures without the test compound or with a placebo are also prepared. The formation of complexes is detected and the formation of complexes in the control reaction but not in the reaction mixture indicates that the test compound interferes with the interaction of the SIGLEC8-L Related Protein and substance. The reactions may be carried out in the liquid phase or the SIGLEC8-L Related Protein, substance, or test compound may be immobilized as described herein. The ability of a compound to modulate the biological activity of a SIGLEC8-L Related Protein of the invention may be tested by determining the biological effects on cells.

It will be understood that the agonists and antagonists i.e. inhibitors and enhancers, that can be assayed using the methods of the invention may act on one or more of the binding sites on the protein or substance including agonist binding sites, competitive antagonist binding sites, non-competitive antagonist binding sites or allosteric sites.

The invention also makes it possible to screen for antagonists that inhibit the effects of an agonist of the interaction of a SIGLEC8-L Related Protein with a substance that is capable of binding to the SIGLEC8-L Related Protein. Thus, the invention may be used to assay for a compound that competes for the same binding site of a SIGLEC8-L Related Protein.

The invention also contemplates methods for identifying compounds that bind to proteins that interact with a SIGLEC8-L Related Protein. Protein-protein interactions may be identified using conventional methods such as co-immunoprecipitation, crosslinking and co-purification through gradients or chromatographic columns. Methods may also be employed that result in the simultaneous identification of genes which encode proteins interacting with a SIGLEC8-L Related Protein. These methods include probing expression libraries with labeled SIGLEC8-L Related Protein.

Two-hybrid systems may also be used to detect protein interactions in vivo. Generally, plasmids are constructed that encode two hybrid proteins. A first hybrid protein consists of the DNA-binding domain of a transcription activator protein fused to a SIGLEC8-L Related Protein, and the second hybrid protein consists of the transcription activator protein's activator domain fused to an unknown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins may be used in the above-described methods. In particular, SIGLEC8-L Related Proteins fused to a glutathione-S-transferase may be used in the methods.

The reagents suitable for applying the methods of the invention to evaluate compounds that modulate a SIGLEC8-L Related Protein may be packaged into convenient kits providing the necessary materials packaged into suitable containers. The kits may also include suitable supports useful in performing the methods of the invention.

4.3 Compositions and Treatments

The proteins of the invention, substances or compounds identified by the methods described herein, antibodies, and antisense nucleic acid molecules of the invention may be used for modulating the biological activity of a SIGLEC8-L Related Protein, and they may be used in the treatment of conditions associated with a SIGLEC8-L Related Protein such as cancer or hematopoietic disorders, in particular aplastic anemia and hematological malignancies such as leukemia and lymphoma, more particularly acute myelogenous leukemia, and chronic myelogenous leukemia.

The substances, antibodies, and compounds may be formulated into pharmaceutical compositions for administration to subjects in a biologically compatible form suitable for administration in vivo. By “biologically compatible form suitable for administration in vivo” is meant a form of the active substance to be administered in which any toxic effects are outweighed by the therapeutic effects. The active substances may be administered to living organisms including humans, and animals. Administration of a therapeutically active amount of a pharmaceutical composition of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, an The compositions are indicated as therapeutic agents either alone or in conjunction with other therapeutic agents or other forms of treatment (e.g. chemotherapy or radiotherapy). For example, the compositions may be used in combination with anti-proliferative agents, antimicrobial agents, immunostimulatory agents, growth factors, cytokines, or anti-inflammatories. In particular, the compounds may be used in combination with anti-viral and/or anti-proliferative agents. The compositions of the invention may be administered concurrently, separately, or sequentially with other therapeutic agents or therapies.

Vectors derived from retroviruses, adenovirus, herpes or vaccinia viruses, or from various bacterial plasmids, may be used to deliver nucleic acid molecules to a targeted organ, tissue, or cell population. Methods well known to those skilled in the art may be used to construct recombinant vectors which will express antisense nucleic acid molecules of the invention. (See, for example, the techniques described in Sambrook et al (supra) and Ausubel et al (supra)).

The nucleic acid molecules comprising full length cDNA sequences and/or their regulatory elements enable a skilled artisan to use sequences encoding a protein of the invention as an investigative tool in sense (Youssoufian H and H F Lodish 1993 Mol Cell Biol 13:98-104) or antisense (Eguchi et al (1991) Annu Rev Biochem 60:631-652) regulation of gene function. Such technology is well known in the art, and sense or antisense oligomers, or larger fragments, can be designed from various locations along the coding or control regions.

Genes encoding a protein of the invention can be turned off by transfecting a cell or tissue with vectors which express high levels of a desired SIGLEC8-Lencoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of a gene encoding a protein of the invention, i.e. the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a protein of the invention.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

An antibody against a SIGLEC8-L Related Protein may be conjugated to chemotherapeutic drugs, toxins, immunological response modifiers, growth factors, cytokines, hematogenous agents, enzymes, and radioisotopes and used in the prevention and treatment of cancer. For example, an antibody against a SIGLEC8-L Related Protein may be conjugated to toxic moieties including but not limited to ricin A, diphtheria toxin, abrin, modeccin, or bacterial toxins from Pseudomonas or Shigella. Toxins and their derivatives have been reported to form conjugates with antibodies specific to particular target tissues, such as cancer or tumor cells in order to obtain specifically targeted cellular toxicity (Moolten F. L. et al, Immun. Rev. 62:47-72, 1982, and Bernhard, M. I. Cancer Res. 43:4420, 1983).

Conjugates can be prepared by standard means known in the art. A number of bifunctional linking agents (e.g. heterobifunctional linkers such as N-succinimidyl-3-(2-pyridyldithio)propionate) are available commercially from Pierce Chemically Company, Rockford, Ill.

Administration of the antibodies or immunotoxins for therapeutic use may be by an intravenous route, although with proper formulation additional routes of administration such as intraperitoneal, oral, or transdermal administration may also be used.

A SIGLEC8-L Related Protein may be conjugated to chemotherapeutic drugs, toxins, immunological response modifiers, enzymes, and radioisotopes using methods known in the art.

The invention also provides immunotherapeutic approaches for preventing or reducing the severity of a cancer. The clinical signs or symptoms of the cancer in a subject are indicative of a beneficial effect to the patient due to the stimulation of the subject's immune response against the cancer. Stimulating an immune response refers to inducing an immune response or enhancing the activity of immunoeffector cells in response to administration of a vaccine preparation of the invention. The prevention of a cancer can be indicated by an increased time before the appearance of cancer in a patient that is predisposed to developing cancer due for example to a genetic disposition or exposure to a carcinogenic agent. The reduction in the severity of a cancer can be indicated by a decrease in size or growth rate of a tumor.

Vaccines can be derived from a SIGLEC8-L Related Protein, peptides derived therefrom, or chemically produced synthetic peptides, or any combination of these molecules, or fusion proteins or peptides thereof. The proteins, peptides, etc. can be synthesized or prepared recombinantly or otherwise biologically, to comprise one or more amino acid sequences corresponding to one or more epitopes of a tumor associated protein. Epitopes of a tumor associated protein will be understood to include the possibility that in some instances amino acid sequence variations of a naturally occurring protein or polypeptide may be antigenic and confer protective immunity against cancer or anti-tumorigenic effects. Sequence variations may include without limitation, amino acid substitutions, extensions, deletions, truncations, interpolations, and combinations thereof. Such variations fall within the scope of the invention provided the protein containing them is immunogenic and antibodies against such polypeptide cross-react with naturally occurring SLG Related Protein to a sufficient extent to provide protective immunity and/or anti-tumorigenic activity when administered as a vaccine.

The proteins, peptides etc, can be incorporated into vaccines capable of inducing an immune response using methods known in the art. Techniques for enhancing the antigenicity of the proteins, peptides, etc. are known in the art and include incorporation into a multimeric structure, binding to a highly immunogenic protein carrier, for example, keyhole limpet hemocyanin (KLH), or diptheria toxoid, and administration in combination with adjuvants or any other enhancer of immune response.

Vaccines may be combined with physiologically acceptable media, including immunologically acceptable diluents and carriers as well as commonly employed adjuvants such as Freund's Complete Adjuvant, saponin, alum, and the like.

It will be further appreciated that anti-idiotype antibodies to antibodies to SIGLEC8-L Related Proteins described herein are also useful as vaccines and can be similarly formulated.

The administration of a vaccine in accordance with the invention, is generally applicable to the prevention or treatment of cancers.

The administration to a patient of a vaccine in accordance with the invention for the prevention and/or treatment of cancer can take place before or after a surgical procedure to remove the cancer, before or after a chemotherapeutic procedure for the treatment of cancer, and before or after radiation therapy for the treatment of cancer and any combination thereof. The cancer immunotherapy in accordance with the invention would be a preferred treatment for the prevention and/or treatment of cancer, since the side effects involved are substantially minimal compared with the other available treatments e.g. surgery, chemotherapy, radiation therapy. The vaccines have the potential or capability to prevent cancer in subjects without cancer but who are at risk of developing cancer.

The activity of the proteins, substances, compounds, antibodies, nucleic acid molecules, agents, and compositions of the invention may be confirmed in animal experimental model systems. Therapeutic efficacy and toxicity may be determined by standard pharmaceutical procedures in cell cultures or with experimental animals, such as by calculating the ED50 (the dose therapeutically effective in 50% of the population) or LD50 (the dose lethal to 50% of the population) statistics. The therapeutic index is the dose ratio of therapeutic to toxic effects and it can be expressed as the ED50/LD50 ratio. Pharmaceutical compositions which exhibit large therapeutic indices are preferred.

4.4 Other Applications

The nucleic acid molecules disclosed herein may also be used in molecular biology techniques that have not yet been developed, provided the new techniques rely on properties of nucleotide sequences that are currently known, including but not limited to such properties as the triplet genetic code and specific base pair interactions.

The invention also provides methods for studying the function of a polypeptide of the invention. Cells, tissues, and non-human animals lacking in expression or partially lacking in expression of a nucleic acid molecule or gene of the invention may be developed using recombinant expression vectors of the invention having specific deletion or insertion mutations in the gene. A recombinant expression vector may be used to inactivate or alter the endogenous gene by homologous recombination, and thereby create a deficient cell, tissue, or animal.

Null alleles may be generated in cells, such as embryonic stem cells by deletion mutation. A recombinant gene may also be engineered to contain an insertion mutation that inactivates the gene. Such a construct may then be introduced into a cell, such as an embryonic stem cell, by a technique such as transfection, electroporation, injection etc. Cells lacking an intact gene may then be identified, for example by Southern blotting, Northern Blotting, or by assaying for expression of the encoded protein using the methods described herein. Such cells may then be fused to embryonic stem cells to generate transgenic non-human animals deficient in a protein of the invention. Germline transmission of the mutation may be achieved, for example, by aggregating the embryonic stem cells with early stage embryos, such as 8 cell embryos, in vitro; transferring the resulting blastocysts into recipient females and; generating germline transmission of the resulting aggregation chimeras. Such a mutant animal may be used to define specific cell populations, developmental patterns and in vivo processes, normally dependent on gene expression.

The invention thus provides a transgenic non-human mammal all of whose germ cells and somatic cells contain a recombinant expression vector that inactivates or alters a gene encoding a SIGLEC8-L Related Protein. In an embodiment the invention provides a transgenic non-human mammal all of whose germ cells and somatic cells contain a recombinant expression vector that inactivates or alters a gene encoding a SIGLEC8-L Related Protein resulting in a SIGLEC8-L Related Protein associated pathology. Further the invention provides a transgenic non-human mammal which does not express or partially expresses a SIGLEC8-L Related Protein of the invention. In an embodiment, the invention provides a transgenic non-human mammal which doe not express or partially expresses, a SIGLEC8-L Related Protein of the invention resulting in a SIGLEC8-L Related Protein associated pathology. A SIGLEC8-L Related Protein pathology refers to a phenotype observed for a SIGLEC8-L Related Protein homozygous or heterozygous mutant.

A transgenic non-human animal includes but is not limited to mouse, rat, rabbit, sheep, hamster, dog, cat, goat, and monkey, preferably mouse.

The invention also provides a transgenic non-human animal assay system which provides a model system for testing for an agent that reduces or inhibits a pathology associated with a SIGLEC8-L Related Protein, preferably a SIGLEC8-L Related Protein associated pathology, comprising:

(a) administering the agent to a transgenic non-human animal of the invention; and

(b) determining whether said agent reduces or inhibits the pathology (e.g. SIGLEC8-L Related Protein associated pathology) in the transgenic non-human animal relative to a transgenic non-human animal of step (a) which has not been administered the agent.

The agent may be useful in the treatment and prophylaxis of conditions such as cancer as discussed herein. The agents may also be incorporated in a pharmaceutical composition as described herein.

The following non-limiting example is illustrative of the present invention:

### EXAMPLE

Materials and Methods

Materials and Methods

Identification of the Genomic Area containing Siglec8

Genomic DNA sequences derived from BAC clones covering chromosome 19q13.4 were identified and obtained from the Lawrence Livermore National Laboratory (LLNL) Human Genome Center. These sequences were compared to the mRNA sequence for Siglec8 (GenBank Accession No. AF195092), which has been reported to be linked to this area (11), using the BLASTN nucleotide alignment tool (22). In addition, genomic regions found to match Siglec8 were also analyzed by the Grail exon prediction program (23), in order to determine the existence of any new Siglecs, as well as possible additional exons for Siglec8. Prediction results were compared to the human EST database by the BLAST alignment tool (22). Further, the genomic region containing Siglec8 was localized to a specific region of chromosome 19q13.4 through the aid of the WebCutter restriction analysis program and comparison of the fragments to the previously published EcoRl map for chromosome 19q13.4 (24).

Molecular Characterization of Siglec8-L

Based on the results of exon prediction and the known sequence of the Siglec8 mRNA, PCR primers were designed to determine the sequence of the Siglec8-L mRNA, as well as to confirm the published Siglec8 nRNA sequence, both through RT-PCR. The primers used were: S8-Forward (common), ACAAGTGACACTGGCAGCAG; S8-L-Reverse, AGCTGAGGGTTGCATAATGG; S8-L-Reverse2, TACTGCATAGCATGGGGCTC; S8-Reverse, AGAAGAGCAGGGGAAACCAC. (SEQ ID Nos 11 to 14.) The fetal liver cDNA used was prepared as described elsewhere (12). The PCR conditions were as follows: 2.5 units of HotStarTaq polymerase (Qiagen, Valencia, Calif.), 1× PCR buffer with 1.5 mM MgCl2 (Qiagen), 1 μL cDNA, 200 μM dNTPs (deoxynucleoside triphosphates), and 250 ng of each primer, using the Mastercycler® gradient thermocycler (Eppendorf Scientific Inc., Westbury, N.Y.). The temperature profile was: denaturation at 95° C. for 15 min. followed by 94° C. for 30 s., annealing at 60° C. for 30 s., and extension at 72° C. for 1 min. for a total of 35 cycles, followed by a final extension at 72° C. for 10 min. The PCR product was subjected to electrophoresis on a 2% agarose gel containing ethidium bromide. The product bands were then extracted from the gel, and the purified DNA was directly sequenced using an automated sequencer.

Marathon-Ready fetal liver cDNA (Clontech, Palo Alto, Calif., USA) was also used to perform nested 3′-RACE in order to verify the 3′ end of the Siglec8 mRNA. The procedure was carried out according to the manufacturer's instructions, with some minor modifications. Briefly, the first round of the 3′ RACE reaction utilized the forward gene-specific primer (GSP1) which is identical to the above mentioned Siglec8-Forward (common) and the provided adapter primer AP1. The nested 3′ RACE reaction was carried out using GSP2 (CCTTCCTGTCCTTCTGCATC) (SEQ ID NO. 15), and the provided AP2. The touchdown PCR method was utilized as recommended by the manufacturer, with a slight temperature profile modification. The annealing temperatures used were: 70° C. for 4 min. for 5 cycles, then 68° C. for 4 min. for 5 cycles, followed by 66° C. for 4 min. for 25 cycles. The denaturation temperature was set to 94° C. for 5 s. for every cycle, with an extension temperature of 72° C. for 1 min. After all cycles were completed a final extension at 72° C. for 10 min. was performed. The reaction was carried out using 2.5 units HotStarTaq (Qiagen, Valencia, Calif.), 1× PCR buffer with 1.5 mM MgCl2 (Qiagen), 200 μM dNTPs, primer concentrations of 200 ng (GSP1 and 2) and 1 μM (AP1 and 2), 1 μL Marathon-Ready cDNA, or 5 μL of the first round 3′ RACE product (50 μL total volume). All 3′ RACE reactions were performed using the Perkin Elmer GeneAmp PCR System 9600 thermocycler (Perkin Elmer, Norwalk, Conn., USA). The products of both rounds of the RACE reaction were subjected to electrophoresis on a 2% agarose gel containing ethidium bromide, with any bands evident being extracted and the DNA directly sequenced with an automated sequencer.

Based on RT-PCR and 3′ RACE results, as well as the initial alignment of the Siglec8 mRNA species to the genomic sequence covering chromosome 19q13.4, the exons of the Siglec8 gene were mapped. This was achieved through the use of the BLASTN nucleotide alignment tool, which enabled localization of the mRNA sequences to specific regions of genomic DNA.

Following final characterization of Siglec8-L, the primary structure of the protein encoded by the Siglec8-L mRNA was determined. This was compared to the published protein sequence for Siglec8, as well as to all the members of the CD33-like subgroup of Siglecs using the CLUSTALX multiple alignment tool (25).

Results

Identification of the Genomic Area containing Siglec8:

The CD33-like subgroup of Siglecs has been mapped, through various means, to chromosome 19q13.4. The Siglec9 gene has recently been characterized and localized to this area (12), immediately following the end of the kallikrein gene family. Further, the mRNA species for the remainder of this subgroup have also been characterized, and mapped to this region primarily through fluorescence in situ hybridization (FISH), as well as somatic cell hybridization (7-11). During examination of this area, a clone, CTD-3073N11, was identified from the CalTech Human BAC library D, that contained the Siglec8 gene. Upon exon prediction analysis of this genomic region, two putative exons were identified at the 3′ terminus of the Siglec8 gene which differed from the previously published mRNA sequence. One of these exons was much shorter in length and the other was not present at all. The human EST database was searched, both with the published mRNA sequence, as well as with the two putative exons, and no significant matches were found.

Based on the sequence information from the clone on which Siglec8 is localized, the WebCutter restriction analysis tool was used to determine the size of the EcoR1 fragments produced. By comparing these results with the available EcoR1 restriction map for chromosome 19q13.4, it was determined that the Siglec8 gene is located in the more centromeric region of 19q13.4, and is approximately 330 kb downstream from the Siglec9 gene.

Molecular Characterization of Siglec8-L

Using RT-PCR and primers derived from sequence shared by both Siglec8 and Siglec8-L, as well as from the 3′ termini of Siglec8 and the putative Siglec8-L, the existence of both putative exons mentioned above was confirmed. Through automated sequencing and subsequent alignment of the mRNA sequence with the genomic sequence for the BAC clone, the exact genomic organization for these last two exons was determined. However, using the PCR primers specific for the published sequence of Siglec8 a very faint band, was obtained after agarose gel electrophoresis, which could not be sequenced. In conjunction with 3′ RACE and the Marathon-Ready fetal liver cDNA additional sequence was identified at the 3′ terminus of Siglec8-L, including both the termination codon and a portion of the 3′ untranslated region. The nested 3′ RACE reaction produced a major product with an approximate size of 300 bp, as well as a much fainter high molecular weight band. Attempts to sequence the latter band were unsuccessful.

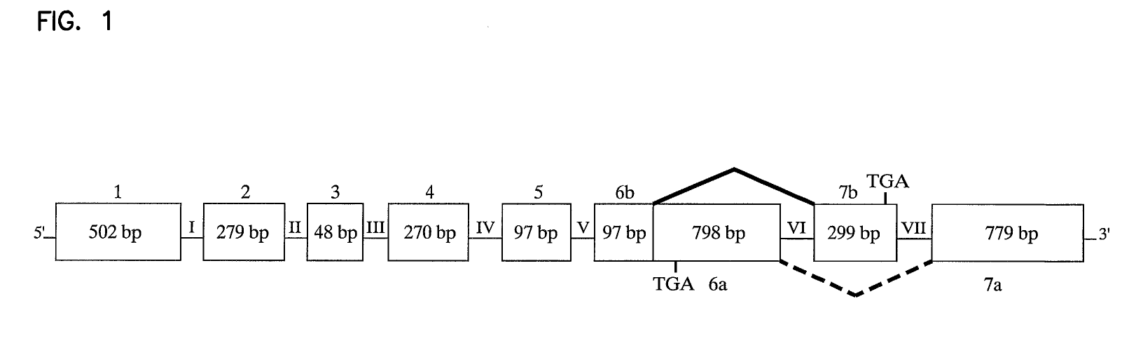

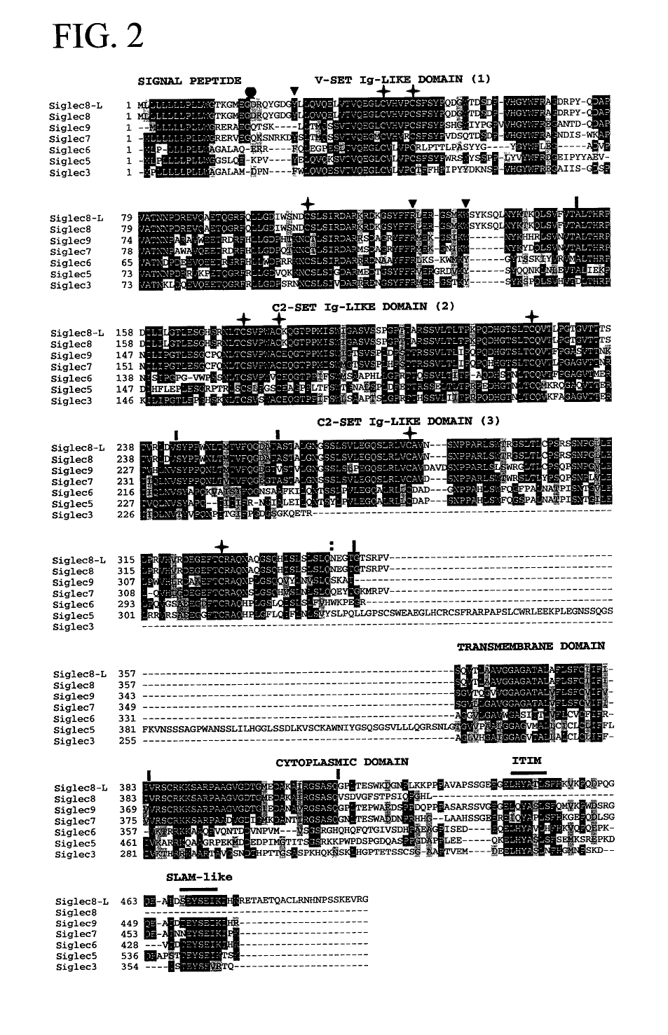

Based on the results obtained through the initial alignment, as well as from the characterization of the last two exons of Siglec8-L, the genomic organization of the Siglec8 gene was characterized. As is shown in FIG. 1, both Siglec8 and Siglec8-L are comprised of seven exons. The first five exons are identical in both Siglec8 and Siglec8-L, with lengths of 502 bp, 279 bp, 48 bp, 270 bp, and 97 bp. The first exon of 502 bp contains a 5′ untranslated region of 48 nucleotides, with the possibility that there is even more upstream sequence. For Siglec8-L, exon six is 97 nucleotides long, while exon seven contains at least 299 bp, of which 252 bp code for amino acid residues, and 44 bp being part of the 3′ untranslated region. Exons six and seven of Siglec8, on the other hand, are 895 bp and 779 bp long, respectively. Examining the splice donor and acceptor sites for each of these exons, it was observed that the first five exons, as well as exons six and seven of Siglec8-L, all possessed sequences closely related to the consensus splice sites (-mGTAAGT . . . CAGm-, where m is any base) (26). However, in the case of Siglec8, these splice sites were not present. Examination of the open reading frame of Siglec8-L revealed a 499 amino acid residue protein with a molecular weight of 54.04 kDa, excluding any post-translational modifications which may be present. The sequence of Siglec8-L is identical to that of Siglec8 until residue 415, after which Siglec8-L contains a sequence homologous to the C-terminal sequences of the CD33-like subgroup of Siglecs, including the two tyrosine-based motifs (FIG. 2).

Discussion

Through efforts to investigate the CD33-like subgroup of Siglecs, the area of chromosome 19q13.4 was identified which contains the Siglec8 gene, located approximately 330 kb downstream of the recently published Siglec9 gene (12). Examination of this area revealed the existence of an alternative form of Siglec8, named Siglec8-L. The protein product of this mRNA species differs markedly from the previously published Siglec8 (GenBank Accession No. AF195092) at its C-terminus (11). Siglec8-L is a 499 residue protein with a molecular weight of 54 kDa. It is encoded by seven exons, and unlike Siglec8, it shows a high degree of homology at its C-terminus to the other members of the CD33-like subgroup of Siglecs. Consistent with its inclusion in this subfamily of Siglecs, Siglec8-L also possesses the two characteristic tyrosine-based motifs.

The Siglec8 mRNA published by Floyd et. al. (2000) contains an abbreviated C-terminus, lacking the characteristic tyrosine-based motifs reported in other members of the CD33-like subgroup of Siglecs (11). Further, based on characterization of its genomic structure, the Siglec8 mRNA species reported by Floyd et. al. contains approximately 1.5 kb of untranslated sequence at its 3′ end. By comparison, and in keeping with the hypothesis that this subgroup of Siglecs arose through gene duplication relatively recently in vertebrate evolution (13, 14), none of the other Siglecs that belong to this subgroup have such an extensive 3′ untranslated region, or any untranslated exons at the 3′ end. Furthermore, the intron/exon splice sites for the last two exons of Siglec8 (based on the genomic sequence and the mRNA sequence of Floyd et. al.) are not consistent with the characteristic splice donor and acceptor sites (26), unlike the first five exons and the two exons identified by us (FIG. 1).

The identification of Siglec8 by Floyd et. al. (2000) was accomplished through the use of an EST that showed homology to CD33 (11). The EST used by Floyd et. al. (11) may represent a partially spliced or incorrectly spliced sequence, which results in the inclusion of two pieces of genomic DNA as well as the absence of an entire exon. During their identification of Siglec8, the authors report an unsuccessful attempt of Northern blot analysis for Siglec8 using a probe derived from the coding and 3′ untranslated sequences of their mRNA species. Based on the problems with ESTs, the lack of specific bands in the Northern blot may be due to such intronic sequence contamination. This contamination likely resulted in a change in the open reading frame of the Siglec8 mRNA which causes premature termination and loss of the two tyrosine-based motifs.

For a few of the other members of the Siglec family, alternative splice forms have been described. For CD22, two isoforms have been identified in humans, with either four or six C2-set Ig-like domains (4, 28, 29). In the mouse there have been three isoforms of MAG identified (30, 31). One of the isoforms lacks an untranslated exon in the 5′ end of the mRNA, which has no effect on the size of the resultant polypeptide. The two other forms differ in the presence of exon 12, which is 45 nucleotides in length and introduces a termination codon when included in the niRNA. In humans, however, there has been no report of any MAG isoforms. Further, two isoforms for CD33 have been reported in the mouse, which differ by an 83 nucleotide in-frame insertion in the cytoplasmic domain (32). Human CD33 has also been reported to exist as two different size transcripts, which is believed to be through the use of alternative polyadenylation signals, with no change in the size of the polypeptide (33). Therefore, although there is evidence of alternative splicing in members of the Siglec family, it appears to occur primarily in non-human members, with the exception of the alternative number of Ig-like domains for human CD22. However, even these cases do not compare to the drastic differences seen in the splicing patterns of Siglec8 and Siglec8-L.

The Siglec family of transmembrane proteins, and in particular the CD33-like subgroup, is a very recently described member of the IgSF (1, 13, 14). Siglec7, identified initially by Falco et. al. (9), was found to be tyrosine-phosphorylated in its ITIM motif, resulting in recruitment of SHP1 with a consequent inhibition of natural killer cell cytotoxicity. More recently, it has been reported that engagement of Siglec7 and CD33, through the use of monoclonal antibodies, inhibits the proliferation of both normal and leukemic myeloid cells in vitro (34). These effects are believed to be the result of phosphorylation of the two tyrosine-based motifs present in the cytoplasmic domain of both CD33 and Siglec7. In addition, Taylor et. al. (35) have found that CD33 recruits the protein-tyrosine phosphatases SHP1 and SHP2, both in vitro and in vivo, and is the result of tyrosine phosphorylation in the ITIM motif. Mutation of the tyrosine in this ITIM motif of CD33 resulted in increased red blood cell binding by CD33-expressing COS cells. These findings suggest that, in addition to the recruitment of SHP1 and 2 inhibiting the activating signaling pathways that lead to cell proliferation and survival, this recruitment may also modulate the receptor's ligand-binding activity (35). It is quite likely, given the high degree of homology within the CD33-like subgroup of Siglecs, that the remainder of this group, including Siglec8-L, play a similar inhibitory role in their respective cell types.

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Crocker, P. R., Clark, E. A., Filbin, M., Gordon, S., Jones, Y., Kehrl, J. H., Kelm, S., Le Douarin, N., Powell, L., Roder, J., Schnaar, R. L., Sgroi, D. C., Stamenkovic, K., Schauer, R., Schachner, M., van den Berg, T. K., van der Merwe, P. A., Watt, S. M., and Varki, A. (1998). Siglecs: a family of sialic-acid binding lectins [letter] Glycobiology 8, v.

2. Crocker, P. R., Kelm, S., Hartnell, A., Freeman, S., Nath, D., Vinson, M., and Mucklow, S. (1996). Sialoadhesin and related cellular recognition molecules of the immunoglobulin superfamily Biochem Soc Trans 24, 150-6.

3. Crocker, P. R., Mucklow, S., Bouckson, V., McWilliam, A., Willis, A. C., Gordon, S., Milon, G., Kelm, S., and Bradfield, P. (1994). Sialoadhesin, a macrophage sialic acid binding receptor for haemopoietic cells with 17 immunoglobulin-like domains Embo J 13,4490-503.

4. Stamenkovic, I., and Seed, B. (1990). The B-cell antigen CD22 mediates monocyte and erythrocyte adhesion Nature 345, 74-7.

5. Ulyanova, T., Blasioli, J., Woodford-Thomas, T. A., and Thomas, M. L. (1999). The sialoadhesin CD33 is a myeloid-specific inhibitory receptor Eur J Immunol 29, 3440-9.

6. Kelm, S., Schauer, R., Manuguerra, J. C., Gross, H. J., and Crocker, P. R. (1994). Modifications of cell surface sialic acids modulate cell adhesion mediated by sialoadhesin and CD22 Glycoconj J 11, 576-85.

7. Cornish, A. L., Freeman, S., Forbes, G., Ni, J., Zhang, M., Cepeda, M., Gentz, R., Augustus, M., Carter, K. C., and Crocker, P. R. (1998). Characterization of siglec-5, a novel glycoprotein expressed on myeloid cells related to CD33 Blood 92, 2123-32.

8. Patel, N., Brinkman-Van der Linden, E. C., Altmann, S. W., Gish, K., Balasubramanian, S., Timans, J. C., Peterson, D., Bell, M. P., Bazan, J. F., Varki, A., and Kastelein, R. A. (1999). OB-BP1/Siglec-6. a leptin- and sialic acid-binding protein of the immunoglobulin superfamily J Biol Chem 274, 22729-38.

9. Falco, M., Biassoni, R., Bottino, C., Vitale, M., Sivori, S., Augugliaro, R., Moretta, L., and Moretta, A. (1999). Identification and molecular cloning of p75/AIRM1, a novel member of the sialoadhesin family that functions as an inhibitory receptor in human natural killer cells J Exp Med 190, 793-802.

10. Nicoll, G., Ni, J., Liu, D., Klenerman, P., Munday, J., Dubock, S., Mattei, M. G., and Crocker, P. R. (1999). Identification and Characterization of a Novel Siglec, Siglec-7, Expressed by Human Natural Killer Cells and Monocytes J Biol Chem 274, 34089-34095.

11. Floyd, H., Ni, J., Cornish, A. L., Zeng, Z., Liu, D., Carter, K. C., Steel, J., and Crocker, P. R. (2000). Siglec-8. A novel eosinophil-specific member of the immunoglobulin superfamily J Biol Chem 275, 861-6.

12. Foussias, G., Yousef, G. M., and Diamandis, E. P. (2000). Identification and Molecular Characterization of a Novel Member of the SIGLEC family Genomics.

13. Angata, T., and Varki, A. (2000). Cloning, characterization and phylogenetic analysis of Siglec-9, a new member of the CD33-related group of Siglecs. Evidence for co-evolution with sialic acid synthesis pathways J Biol Chem.

14. Zhang, J. Q., Nicoll, G., Jones, C., and Crocker, P. R. (2000). Siglec-9. A novel sialic acid binding member of the immunoglobulin superfamily expressed broadly on human blood leukocytes J Biol Chem.

15. Burshtyn, D. N., Yang, W., Yi, T., and Long, E. O. (1997). A novel phosphotyrosine motif with a critical amino acid at position −2 for the SH2 domain-mediated activation of the tyrosine phosphatase SHIP-1 J Biol Chem 272, 13066-72.

16. Vivier, E., and Daeron, M. (1997). hnmunoreceptor tyrosine-based inhibition motifs Immunol Today 18,286-91.

17. Borges, L., Hsu, M. L., Fanger, N., Kubin, M., and Cosman, D. (1997). A family of human lymphoid and myeloid Ig-like receptors, some of which bind to MHC class I molecules J Immunol 159, 5192-6.

18. Le Drean, E., Vely, F., Olcese, L., Cambiaggi, A., Guia, S., Krystal, G., Gervois, N., Moretta, A., Jotereau, F., and Vivier, E. (1998). Inhibition of antigen-induced T cell response and antibody-induced NK cell cytotoxicity by NKG2A: association of NKG2A with SHP-1 and SHP-2 protein-tyrosine phosphatases [published erratum appears in Eur J Immunol 1998 March;28(3): 1122] Eur J Immunol 28, 264-76.

19. Muraille, E., Bruhns, P., Pesesse, X., Daeron, M., and Emeux, C. (2000). The SH2 domain containing inositol 5-phosphatase SHIP2 associates to the immunoreceptor tyrosine-based inhibition motif of Fc gammaRIIB in B cells under negative signaling Immunol Lett 72, 7-15.

20. Coffey, A. J., Brooksbank, R. A., Brandau, O., Oohashi, T., Howell, G. R., Bye, J. M., Cahn, A. P., Durham, J., Heath, P., Wray, P., Pavitt, R., Wilkinson, J., Leversha, M., Huckle, E., Shaw-Smith, C. J., Dunham, A., Rhodes, S., Schuster, V., Porta, G., Yin, L., Serafini, P., Sylla, B., Zollo, M., Franco, B., Bentley, D. R., and et al. (1998). Host response to EBV infection in X-linked lymphoproliferative disease results from mutations in an SH2-domain encoding gene [see comments] Nat Genet 20, 129-35.

21. Sayos, J., Wu, C., Morra, M., Wang, N., Zhang, X., Allen, D., van Schaik, S., Notarangelo, L., Geha, R., Roncarolo, M. G., Oettgen, H., De Vries, J. E., Aversa, G., and Terhorst, C. (1998). The X-linked lymphoproliferative-disease gene product SAP regulates signals induced through the co-receptor SLAM [see comments] Nature 395, 462-9.

22. Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs Nucleic Acids Res 25, 3389-402.

23. Murakami, K., and Takagi, T. (1998). Gene recognition by combination of several gene-finding programs Bioinformatics 14, 665-75.

24. Ashworth, L. K., Batzer, M. A., Brandriff, B., Branscomb, E., de Jong, P., Garcia, E., Garnes, J. A., Gordon, L. A., Lamerdin, J. E., Lennon, G., and et al. (1995). An integrated metric physical map of human chromosome 19 Nat Genet 11, 422-7.

25. Jeanmougin, F., Thompson, J. D., Gouy, M., Higgins, D. G., and Gibson, T. J. (1998). Multiple sequence alignment with Clustal X Trends Biochem Sci 23, 403-5.

26. Iida, Y. (1990). Quantification analysis of 5′-splice signal sequences in mRNA precursors. Mutations in 5′-splice signal sequence of human beta-globin gene and beta-thalassemia J Theor Biol 145, 523-33.

27. Wolfsberg, T. G., and Landsman, D. (1997). A comparison of expressed sequence tags (ESTs) to human genomic sequences Nucleic Acids Res 25, 1626-32.

28. Wilson, G. L., Fox, C. H., Fauci, A. S., and Kehrl, J. H. (1991). cDNA cloning of the B cell membrane protein CD22: a mediator of B-B cell interactions J Exp Med 173, 137-46.

29. Wilson, G. L., Najfeld, V., Kozlow, E., Menniger, J., Ward, D., and Kehrl, J. H. (1993). Genomic structure and chromosomal mapping of the human CD22 gene J Immunol 150, 5013-24.

30. Fujita, N., Sato, S., Kurihara, T., Kuwano, R., Sakimura, K., Inuzuka, T., Takahashi, Y., and Miyatake, T. (1989). cDNA cloning of mouse myelin-associated glycoprotein: a novel alternative splicing pattern Biochem Biophys Res Commun 165, 1162-9.

31. Fujita, N., Sato, S., Kurihara, T., Inuzuka, T., Takahashi, Y., and Miyatake, T. (1988). Developmentally regulated alternative splicing of brain myelin- associated glycoprotein mRNA is lacking in the quaking mouse FEBS Lett 232, 323-7.

32. Tchilian, E. Z., Beverley, P. C., Young, B. D., and Watt, S. M. (1994). Molecular cloning of two isoforms of the murine homolog of the myeloid CD33 antigen Blood 83, 3188-98. 33. Simmons, D., and Seed, B. (1988). Isolation of a cDNA encoding CD33, a differentiation antigen of myeloid progenitor cells J Immunol 141, 2797-800.

34. Vitale, C., Romagnani, C., Falco, M., Ponte, M., Vitale, M., Moretta, A., Bacigalupo, A., Moretta, L., and Mingari, M. C. (1999). Engagement of p75/AIRM1 or CD33 inhibits the proliferation of normal or leukemic myeloid cells Proc Natl Acad Sci U S A 96, 15091-6. 35. Taylor, V. C., Buckley, C. D., Douglas, M., Cody, A. J., Simmons, D. L., and Freeman, S. D. (1999). The myeloid-specific sialic acid-binding receptor, CD33, associates with the protein-tyrosine phosphatases, SHP-1 and SHP-2 J Biol Chem 274, 11505-12.

36. Pedraza, L., Owens, G. C., Green, L. A., and Salzer, J. L. (1990). The myelin-associated glycoproteins: membrane disposition, evidence of a novel disulfide linkage between immunoglobulin-like domains, and posttranslational palmitylation J Cell Biol 111, 2651-61.

37. Williams, A. F., Davis, S. J., He, Q., and Barclay, A. N. (1989). Structural diversity in domains of the immunoglobulin superfamily Cold Spring Harb Symp Quant Biol 54, 637-47.

38. May, A. P., Robinson, R. C., Vinson, M., Crocker, P. R., and Jones, E. Y. (1998). Crystal structure of the N-terminal domain of sialoadhesin in complex with 3′ sialyllactose at 1.85 A resolution Mol Cell 1, 719-28.

39. Nielsen, H., Engelbrecht, J., Brunak, S., and von Heijne, G. (1997). A neural network method for identification of prokaryotic and eukaryotic signal peptides and prediction of their cleavage sites Int J Neural Syst 8, 581-99.

40. Williams, A. F., and Barclay, A. N. (1988). The immunoglobulin superfamily—domains for cell surface recognition Annu Rev Immunol 6, 381-405.

We claim:

1. An isolated nucleic acid molecule which comprises:
(i) a nucleic acid sequence encoding a protein having 90% sequence identity with the amino acid sequence shown in SEQ. ID. NO 10;
(ii) nucleic acid sequences complementary to (i); or
(iii) a degenerate form of a nucleic acid sequence of (i).

2. An isolated nucleic acid molecule as claimed in claim 1 which comprises:
(a) a nucleic acid sequence having 90% sequence identity or sequence similarity with a nucleic acid sequence of one of SEQ. ID. NOs. 1, 7, 8, or 9;
(b) nucleic acid sequences complementary to (i), preferably complementary to the full nucleic acid sequence of one of SEQ. ID. NOs. 1, 7, 8, or 9; or
(c) nucleic acid sequences differing from any of the nucleic acid sequences of (i) or (ii) in codon sequences due to the degeneracy of the genetic code.

3. A vector comprising a nucleic acid molecule of claim 1.

4. A host cell comprising a nucleic acid molecule of claim 1.

5. An isolated SIGLEC8-L protein comprising an amino acid sequence of SEQ. ID. NO. 10.

6. A method for preparing a SIGLEC8-L protein comprising:
(a) transferring a vector as claimed in claim 3 into a host cell;
(b) selecting transformed host cells from untransformed host cells;
(c) culturing a selected transformed host cell under conditions which allow expression of the protein; and
(d) isolating the protein.

7. An antibody having specificity against an epitope of a protein as claimed in claim 5.

8. A probe comprising a sequence encoding a protein as claimed in claim 5.

9. A method of diagnosing and monitoring a condition associated with a SIGLEC8-L protein by determining the presence of a nucleic acid molecule as claimed in claim 1.

10. A method of diagnosing and monitoring a condition associated with a SIGLEC8-L protein by determining the presence of a protein as claimed in claim 5.

11. A method for identifying a substance which associates with a protein as claimed in claim 5 comprising (a) reacting the protein with at least one substance which potentially can associate with the protein, under conditions which permit the association between the substance and protein, and (b) removing or detecting protein associated with the substance, wherein detection of associated protein and substance indicates the substance associates with the protein.

12. A method for evaluating a compound for its ability to modulate the biological activity of a protein as claimed in claim 5 comprising providing a known concentration of the protein with a substance which associates with the protein and a test compound under conditions which permit the formation of complexes between the substance and protein, and removing and/or detecting complexes.

13. A method for detecting a nucleic acid molecule encoding a SIGLEC8-L protein in a biological sample comprising the steps of:
(a) hybridizing a nucleic acid molecule of claim 1 to nucleic acids of the biological sample, thereby forming a hybridization complex; and
(b) detecting the hybridization complex wherein the presence of the hybridization complex correlates with the presence of a nucleic acid molecule encoding the protein in the biological sample.

14. A method for treating a condition mediated by a SIGLEC8-L protein comprising administering an effective amount of an antibody as claimed in claim 7.

15. A composition comprising a protein claimed in claim 5.

16. A composition comprising a compound identified using a method as claimed in claim 12, and a pharmaceutically acceptable carrier, excipient or diluent.

17. A transgenic non-human mammal which does not express or partially expresses a SIGLEC8-L protein as claimed in claim 5 resulting in a SIGLEC8-L associated pathology.
